(19) United States
(12) Patent Application Publication
(10) Pub. No.: US 2001/0023355 A1
(43) Pub. Date: Sep. 20, 2001

(54) Ear piercing healing grommet system

(76) Inventor: Steve Danno, Laguna Hills, CA (US)

Correspondence Address: Matthew A. Newboles, STETINA BRUNDA GARRED & BRUCKER, 75 Enterprise, Suite 250, Aliso Viejo, CA, 92656, US

(21) Appl. No.: 09/747,128

(22) Filed: Dec. 22, 2000

Related U.S. Application Data
(63) Continuation-in-part of application No. 09/264,498, filed on Mar. 8, 1999, now Pat. No. 6,183,490.

(51) Int. Cl. A61B 17/34

(52) U.S. Cl. 606/188

(57) ABSTRACT

An improved ear piercing system having a healing grommet with a cannula member detachably mounted thereon. In such embodiment the grommet may be formed of a flexible bio-compatible material such silicone and may be formed to have self anchoring properties. The grommet is formed with a collar or flange member at the proximal end allowing the grommet to be easily grasped and rotated intermittently as is critical to healing. The grommet allows individuals to change jewelry namely earrings or hoops immediately following piercing, and throughout the extended healing process.

### CROSS-REFERENCE TO RELATED APPLICATIONS

This Application is a continuation-in-part of U.S. patent application Ser. No. 09/264,498 filed Mar. 8, 1999.

### STATEMENT RE: FEDERALLY SPONSORED RESEARCH/DEVELOPMENT

(Not Applicable)

### BACKGROUND OF THE INVENTION

It is well known and well publicized that infections and problems associated with ear piercing continue to be widespread as they have remained so for decades. It is additionally well known in the industry that the sale of jewelry is the primary object of the dominant vendors engaged in such business, and that the ear piercing service is a means to the primary end insofar as the piercing service is frequently offered “free” with the purchase of jewelry.

While the pierced openings of most end user consumers eventually heal, it is well known that a significant number of piercings do not heal optimally, and that a significant number sustain unnecessarily long term recurring discomfort traceable to the original piercing. Given the absence of viable choice in the marketplace, consumers, many of which are young children, often must accept the only standard of service available. Such reluctant participation even occurs despite the fact that parents remain extremely suspicious and concerned about the quality of piercing being practiced at most retail sites, and that parents of increasingly younger end user consumers would greatly appreciate more affordable choices that address their serious consumer issues.

To deal with ear piercing problems effectively and comprehensively, two well known industry dynamics must be addressed. The first dynamic includes the marketing practices and incentives of the current providers which are frequently at cross purposes with optimum piercing and healing. It is well known that the dominant vendors have strong incentives to sell earrings, especially gold earrings with standard diameter (small diameter) posts at the point of sale and the time of piercing.

For example, one well known piercing practice critically advantageous to the consumer is to employ a large diameter piercing post, and to utilize medical grade standards. It is well known in the industry that the proper piercing post to utilize will optimally be formed of non-reactive hypoallergenic metal void of nickel (i.e., medical grade stainless steel or titanium). It is likewise well known that the piercing shaft (about 0.048″ diameter) that is approximately twice the diameter of the posts of standard ornamental earrings (about 0.028″ diameter) is further advantageous to optimum piercing.

Notwithstanding such safeguards, even after the ear is initially healed, the walls of the pierced opening are still tender and vulnerable to scratching when changing earrings. Such scratching promotes the opportunity for infection and delays the healing process. When changing out earrings after initial healing the end user consumer is much less likely to scratch and infect the tender walls of the pierced opening if a larger opening has been initially formed during healing. Moreover, it is well understood in the industry that the newly pierced opening will gradually close to the diameter of the smallest shaft diameter inserted. It is also well known in the industry that the preferable large diameter piercing earring must be discarded after healing. (After the opening closes to the small diameter of ornamental jewelry the large diameter piercing post would itself tend to scratch the newly reduced opening.)

The dominant industry vendors are also known to offer the above options of large diameter stainless steel piercing earrings in their product lines. However, in practice the well known market trend has been to promote the sale of the much more profitable small diameter ornamental gold piercing earrings throughout the sales and distribution channels. For example, sales at the point of purchase commonly stall when consumers learn or are reminded that the piercing earring must remain in place for about six continual weeks, day and night. To overcome this and other consumer objections, it is well known in the industry that in order to promote the retail sale of gold earrings at the points of piercing, consumers are commonly informed by retail clerks: (1) that the small diameter shaft is adequate; (2) that gold is hypoallergenic and non reactive and adequate for the healing phase; (3) that by piercing with (standard diameter) ornamental gold earring the customer can conserve their investment by having an earring they can use after piercing versus having to throw away the large diameter piercing earring; and (4) that consumers can likely change out their earrings to standard earrings as early as four weeks versus the standard six weeks of healing.

The second well known dynamic affecting optimal healing is consumer behavior. Consumers, especially younger consumers, have a strong inclination or impatience to change out earrings before healing is complete. Such impatience, combined with the above-noted “permission” at the point of sale or piercing, leads too commonly to changing earrings prematurely. Invariably, while the walls of the pierced ear are still tender, consumers insert standard earrings of unknown metallic content into the small diameter opening. Frequently, the walls of the small opening are scratched, and/or the exchanged earring may not have been properly cleaned. Commonly infections and extensive delays to healing occur, not to mention other problems, often long term. A critical time period for the consumer is the last two weeks of the healing period. Hence, the strong impatience of the consumer to change out their piercing earring is a major factor relating to infections during this period. The products and services of the dominant vendors do not adequately address the above consumer behavioral factor. As mentioned, to do so would be deemed to be at cross purposes with sale of more profitable earrings.

A piercing system designed to comprehensively address consumer health issues cannot avoid addressing the above consumer behavioral factors. Hence, what is greatly needed in the industry is a means of completely designing around the consumers' behavioral impatience to change out earrings. As such, generally speaking a piercing/healing grommet implanted at the time of piercing would provide the means for the consumer to interchangeably insert ornamental earrings throughout the healing period. The consumer would gain every reason to be patient during the last critical weeks of healing. The tender newly pierced opening would remain insulated from any earring post introduced inside the grommet. The grommet diameter would be optimum as well, as described above.

While devices have been developed that are directed to sleeves and other like devices for facilitating the wearing of jewelry, the same are directed to use following a completely healed piercing. Exemplary of such devices include those disclosed in U.S. Pat. No. 4,067,341 to Ivey; U.S. Pat. No. 4,593,540 to Cuvar; U.S. Pat. No. 4,829,788 to DiDomenico; U.S. Pat. No. 5,018,365 to Luceno; U.S. Pat. No. 5,154,068 to DiDomenico; and U.S. Pat. No. 5,743,113 to Kogen.

For the following reasons all the referenced patents share the similar distinction of being restricted by design and intent to application after the pierced channel in the ear has healed.

1) The referenced patents are self defined and self limiting to after piercing and after healing applications. The focus of each patent is to protect against the common allergic reaction to low grade materials common to standard ornamental earrings, or to protect against irritation or scratching from repeated penetration caused by changing out earrings after the ear has been pierced and healed. None of the patents make any mention to their products as facilitating during healing. None of the referenced patents make any mention of insertion of a grommet, or means of insertion, simultaneous to piercing. The designs of the referenced patents necessarily locks in and restricts them to after-healed applications. Such designs, however subtle, are counter-productive and often mutually exclusive to the design of a grommet designed for the healing phase. For example:

The need to rotate the shaft during healing: A healing grommet requires a sturdy flange designed for easy and regular gripping by the fingers for rotation purposes. And, such rotation requires easy access (provided by a small gap from the earlobe) to allow the fingers room to grip the flange. Yet, the collars or flanges of the referenced patents are intentionally and usually designed to be smaller than the jewelry to remain hidden or camouflaged. And, decorative (i.e., star-shaped) flanges are conducive to decoration and not for gripping to rotate. Furthermore, the above patents all refer to “abutting the flanges snugly (sandwiching) against the earlobe”. Such abutment is counter-productive to easy access for gripping and rotating. The rear flanges in the above list have inherent designs that are further counter productive to the above rotation. Yet, the rear flanges on the above lists are intended to be press fit and not positively linked to the shaft. Otherwise, they are snapped into axial channels (as with Kogen) so the flange will tend to rotate without turning the shaft post.

A gap for air flow is critical to healing: During healing some lateral longitudinal “looseness” between the healing post and an air inlet is critical for at least three reasons. a) As already mentioned, lateral spacing between the flange and the earlobe is needed to have ready access to grip the flange for rotation. b) Such spacing is also critical for the free flow of cleaning solutions during regular washing. And c) spacing is critical to let oxygen gain access to the healing wound Yet, as mentioned, such spacing is counter productive to the design of all the listed patents which focus upon “snugly abutting” the flanges for permanent or semi-permanent attachment.

Alignment of the pierced opening is critical to healing grommets: With healing grommets it is critical that the shortest possible channel be pierced across the earlobe. The pierced distance is critical because of the following dual situation: a) the need for the above-mentioned gap/looseness; and b) the need to maintain the space to “catch” and retain the distal end of the earring post or hoop in a clasp or clutch. The above listed patents make no mention of special piercing devices. Conventional piercing devices are all aligned on one internal track. Yet, none of the conventional devices positively clamp (“lock”) onto the earlobe, and due to the resulting “play” (looseness) around the earlobe target at the moment of piercing, conventional piercing is prone to human error in piercing a less than perpendicular (shortest) path across the earlobe. And, none of the listed patents adequately address the issue of piercing alignment.

Healing shafts need to be smooth: During healing it is critical to avoid pockets or cavities along the shaft due to the potential accumulation of bacteria. (Ideally, the material selected for the healing shaft or sleeve will be a hypoallergenic material the body will not “grow to” such as silicone.). Yet, at least two of the patents in the above list offer shafts with such cavities coming in contact with the earlobe opening. Kogen's. And, Luceno provides for threads for threading an end cap. The threads design requires a longitudinal slot extending midway along the shaft to provide for a necessary spring tension necessarily encroach into the ear opening because Luceno recommends “abutting the earlobe front and rear”.

Healing grommets are primarily intended for use during the healing period: Yet the above listed patents are admittedly designed for after healing and with permanence or semi-permanence in mind. The above products are designed to be “cleanable and re-usable” (Kogen).

Healing grommets need to be of simple design and disposable: Yet, most of the referenced designs are of complex and expensive construction.

The only relevant prior art known to Applicant comprises a piercing grommet that was introduced and patented by S. Thomas U.S. Pat. No. 5,411,516 issued May 2, 1995. While it may be shown to have certain drawbacks, the Thomas design serves as a revolutionary early step taken to comprehensively address and solve sustained consumer health issues in ear piercing.

Thomas provides for a piercing and healing grommet he refers to as a hollow post which allows for the interchange of earrings throughout healing. During piercing a solid sharpened post having a sharp point is inserted into the hollow post to form one piercing assembly. Using conventional piercing guns provided by the dominant vendors, the piercing assembly is forced through the earlobe tissue, forming the desired opening. The solid post is removed and the hollow post is retained at the rear by a standard “butterfly” clutch. The hollow post (grommet) then remains implanted throughout the approximate six week healing phase. In the Thomas design the front of the hollow tube post is specialized to form a cup. A rubber plug with a bored opening is lodged into the cup for the purpose of receiving and frictionally holding the posts of standard earrings. The cup/plug housing has ample depth to stand off of the ear and serve the dual purpose of a grasping means to provide for the necessary periodic rotation of the grommet post for proper healing to take place.

Certain drawbacks of the Thomas design are noted. From a cosmetic perspective, consumers are conventionally accustomed to earrings residing adjacent to the earlobe or at least appearing to butt against the earlobe. The effect of Thomas' substantial cup/plug housing would cause the earring to stand off from the earlobe and would be strongly unconventional and predictably undesirable to consumers. The Thomas patent claims to retain the conventional earring posts “within the hollow post” (grommet). However Thomas also states that the earring posts are retained “within the rubber plug”; and that the central bore of the rubber plug is smaller than the central bore of the hollow carry-through post. It appears that Thomas clearly intends for the earring post to be frictionally retained externally such as by the rubber plug and not frictionally held by the interior walls of the grommet's hollow post or shaft.

Secondly, the hollow post grommet of Thomas' design is understood to be made of a very rigid material such as metal in all contemplated embodiments, as its initial function is to be used as the piercing device in assembly or by itself. A cutting edge is conventionally expected to be formed or ground. A curved shaped hollow tube is not conducive to the solid carry-through post and piercing gun method described. As such, the grommet in the Thomas patent is intended to accommodate straight earrings, and not serve a dual purpose of being curved to receive and retain hoop shaped earrings.

Thirdly, in the Thomas design an incision cutting edge is preferably formed by the joining of two components, the hollow post and the solid carry-through post. The beveled and sharpened ends are intended to nest to form one sharpened point the purpose being to minimize or nearly eliminate any gap where the two parts meet. However it would be remarkably difficult to maintain the tolerances necessary to substantially eliminate the said joint or gap. It would be undesirable to have a gap of any size occur along the incision point. There are multiple opportunities during production, assembly and operation of the device to deviate from holding ideal precise tolerances. Maintaining a smooth joint at the distal end would be difficult. Optimally, the ends should be sharpened in the assembled state. Current grinding methods would require additional handling to separate and clean the parts prior to re-assembly, also running the risk of separating matched hollow post and solid carry-through post pairs.

The benefits of specialized sharpened needles (cannulas) are well known in medical arts. It is also well known that such specialization relates directly to a given precise task and to minimizing discomfort and to promote optimum healing. The sharpened point suggested in the Thomas design is severely limited and predetermined by the hollow post and solid carry-through rod assembly. The shape of the leading cutting edge desired to form an opening through an earlobe or through ear cartilage will optimally be more specialized than the piercing point described in the Thomas design. Furthermore, an optimum incision point is one seamless cutting edge rather than two parts joined loosely. Furthermore, the point of Thomas' ear piercing post device bares little difference or improvement over the conventional practice of forcing a pointed standard earing post through ear tissue. It is well known in medical arts that, by not providing a cutting edge below the tip, a tearing effect results, as with forcing a sharp pencil through paper.

Fourthly, the Thomas design offers the option of firing the hollow post by itself through the ear rather than as an assembly. In such case the hollow post would either need to be formed to be closed at the end and then sharpened. Or the hollow post would need to be ground as with a medical grade needle or cannula. In either case the cost could be prohibitively expensive. It is well known that handling very short discretely shaped cannulas for the purpose of grinding and later cleaning are very costly and not amenable to automation. More importantly, using the hollow post alone as the cutting edge would be greatly impractical for additional reasons. It is understood that the healing grommet must remain implanted continually night and day for the duration of the approximate six week healing phase. And it is well known that consumers are reluctant to sleep with earrings as they may compress the rear clutch against the side of the consumer's neck. No reference is made in the Thomas patent to protect the consumer from the sharp end of such sharpened hollow post to mitigate against such noted scratching. No reference is noticed which refers to replacing same hollow piercing post implant.

### SUMMARY OF THE INVENTION

The present invention specifically addresses and alleviates the above identified deficiencies in the art. It is therefore the object of this invention to provide an improved tubular piercing earring or grommet implant, that remains in the ear throughout the extended healing process, having the purpose of allowing the shafts of conventional ornamental earrings and hoops to be interchangeably inserted immediately after piercing.

Another object of this invention is to substantially eliminate most problems resulting from changing earrings or hoops before the pierced opening is fully healed. The invented piercing implant grommet will be formed of a bio-compatible flexible material. Unlike prior art the shaft of the implant grommet will conform to the shape of the inserted ornamental earring shafts or now popular hoops. (Such silicone material is further desirable during healing in that it is well known in medical arts that the body is naturally inclined not to attach to or “grow” to silicone. Importantly the material may be saturated or coated with active anti-microbial agents and germicides.)

Another object of the invention is to implant the grommet while piercing the ear and to provide for an optimum incision that reduces discomfort and facilitates rapid healing. Unlike prior art the present invention will provide for a one-piece incision device, or cannula, with a cutting edge that may be customized to optimally suited to piercing the earlobe or cartridge, and to minimize or prevent tearing during piercing.

Another object of the invention is for the proximal and or distal end of the grommet to be formed as a collar or flange which may be readily grasped for intermittent rotation critical to the healing process.

Another object of the invention is to generally camouflage the above flange or grommet collar from casual view. To this end it is the object of the invention to provide a flange that is not noticeable because it is close to the surface of ear, and because the flange diameter is similar in size or smaller than the ornamental earring or hoop jewelry. The silicone grommet may be molded with durometer hardness and thickness to provide the critical balance between the stiffness required in the flange and the suppleness required in the grommet's hollow shaft. The grommet and its flange may further be formed of translucent or of selectable skintone colors so as to be even less noticeable during use.

Another object of the invention is to maintain the inserted ornamental jewelry close to the ear so such jewelry does not stand off considerably or noticeably as with prior art. With the present invention the proximal collar may have a relatively narrow cross section since the jewelry is retained within the grommet shaft and not retained by an external rubber plug and cup assembly as with prior art.

Another object of the invention is to provide a piercing grommet system that can be deployed by a quick, easy, sterile method with minimum discomfort. Therefore, with the present invention the grommet implant is delivered into the pierced passageway by first being inserted or carried inside a seamless one-piece hollow or closed tube or cannula with a sharpened precision cutting edge. During production the grommet is loaded into the cannula to form a grommet assembly or cartridge, to in turn be loaded into the barrel of a piercing device which will also provide the alignment needed before firing or forcing the cannula assembly accurately into the ear. After piercing, the cannula is automatically and immediately isolated into the catch cartridge of the device on the opposite side of the targeted piercing.

Another object of the invention is to provide a means of retaining the grommet and/or the earring post without scratching the head and neck while reclining or sleeping. Such potential scratching may be completely avoided because the ornamental earring and clasp may be easily removed and because the healing grommet is to be formed of relatively soft and flexible materials. On the contrary, the grommet of prior art is necessarily formed of a hard material with a beveled end and designed to be retained by a conventional clasp.

Another object of the invention is to retain the inserted conventional earring posts or hoops within the grommet shaft portion with a frictional press fit, and not as in prior art by a rubber plug standing off considerably and noticeably outside the pierced channel. Friction may be provided by the close fit of the soft interior walls of the grommet shaft. Friction upon the earring shaft may be indirectly enhanced by the normal compression force of the earlobe or cartilage tissue surrounding the grommet. Optionally, the inserted earring posts may be retained within the grommet by a wedging or “pinching” effect provided by tapering the interior walls of the grommet to form a narrowing channel.

Optionally the earring and the grommet may be retained conventionally by a rear clasp or clutch attached to the ornamental earring or hoop.

Another object of the present invention is to provide for positive retention of the grommet at the distal end to further mitigate the potential problem of removing the grommet when removing the interchangeable ornamental earrings. The distal end of the grommet may be tapered and barbed, or may be circumscribed with a channel or groove. Immediately after piercing, and potentially automatically, a washer or “C” clip may be slid over or around the distal end of the grommet and be retained by the barbed design or within a channel slot.

Another object of the invention is to provide a piercing grommet that is disposable and replaceable during the extended healing process. The grommets may be pre-saturated with anti-microbial germicides. Such germicides remain as active agents for limited time periods. Thus a disposable grommet provides the means of keeping the newly pierced opening clean and maintaining fresh active anti-microbial agents at work in the pierced opening.

Another object of the invention is to provide for replacement grommets described above which are self anchoring. The distal end of such grommets would have larger diameters than the diameter of the pierced channel or the grommet shaft portion. The distal end may be hollow or closed. In use the flexible and compressible distal end will be positioned into a jaw type clamp. The arms of the clamp would compress the grommet end allowing the clamp grommet assembly to be comfortably inserted through the pierced opening. When the jaw type clamp is pulled off the grommet the distal end of the grommet would be free to retain its larger memory shape and thereby be retained at both ends of the pierced channel.

### BRIEF DESCRIPTION OF THE SEVERAL VIEWS OF THE DRAWINGS

These as well as other features of the present invention will become more apparent upon reference to the drawings.

FIG. 1 is a perspective view of the piercing/healing grommet assembly components including a cannula member, and healing grommet in accordance with a preferred embodiment of the present invention within which is nested a conventional earring also shown;

FIG. 2 is a perspective view of a cannula constructed in accordance with a second preferred embodiment of the present invention wherein there is depicted a cannula lumen with a closed sharpened distal end;

FIG. 3 is a perspective view of a healing grommet constructed in accordance with a fourth preferred embodiment of the present invention wherein is depicted a washer retaining system;

FIG. 4 is a perspective view of a healing grommet constructed in accordance with a third preferred embodiment of the present invention wherein is depicted a grommet with a closed, bulbous distal end;

FIG. 5 is a perspective view of a healing grommet constructed in accordance with a second preferred embodiment of the present invention wherein is depicted a grommet with a hollow trumpet shaped distal end;

FIG. 6 is a perspective view of a healing grommet constructed in accordance with a fifth preferred embodiment of the present invention wherein is depicted a textured shaft for resisting inadvertent removal;

FIG. 7 is a cross sectional view of a healing grommet constructed in accordance with a fifth preferred embodiment of the present invention wherein is depicted a means for impinging a standard earring shaft; and

FIG. 8 is a perspective view of a jaw type “clamp” used to compress the distal ends of replacement healing grommets such as those described in FIGS. 4 and 5.

### DETAILED DESCRIPTION OF THE INVENTION

The detailed description as set forth below in connection with the appended drawings is intended as a description of the presently preferred embodiments of the invention, and is not intended to represent the only form in which the present invention may be constructed or utilized. The description sets forth the functions and sequences of steps for constructing and operating the invention in connection with the illustrated embodiments. It is understood, however, that the same or equivalent functions and sequences may be accomplished by different embodiments and that they are also intended to be encompassed within the scope of this invention.

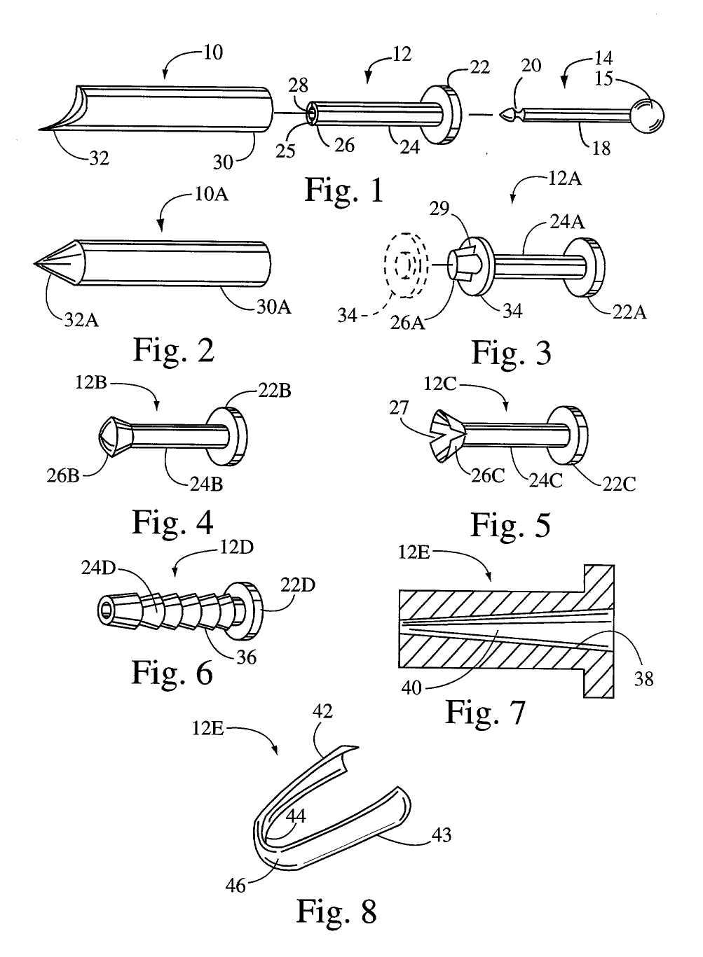

Referring now to the Figures, and initially to FIG. 1, there is shown the components of an improved ear piercing/healing grommet and delivery system. An elongate cylindrical sleeve or healing grommet 12 is formed of a bio-compatible material, such as medical grade silicone. In such embodiment, the sleeve or grommet 12 is specifically designed to embed and reside within the pierced channel opening in the ear. The grommet 12 may be provided with an annular collar or flange 22 formed about the proximal end thereof which serves to radially abut about the tissue surrounding the piercing opening, thereby defining the entryway into the piercing and provide means for anchoring the grommet 12 axially within the formed piercing. As a consequence, grommet 12 will provide the individual with a channel or pathway through which he or she may insert ornamental earrings and the like and change jewelry, which is not available via piercing made directly through flesh insofar as such piercing necessarily require that the tissue heal about such piercing, which can take over a month for initial healing to take place and up to several months for complete healing for most individuals.

In a preferred embodiment the grommet 12 may be formed of a relatively flexible material to thus enable individuals to readily insert and affix earrings or other types of jewelry having arcuate contours, such as hoops and the like. By varying the durometer hardness of various grommet materials such as silicone, and by varying the thickness of various sections of the grommet 12 advantages may be gained over prior art for achieving vital and specialized tasks. For example, it is well known that intermittent rotation of the designated imbedded piercing shaft (or in this case a healing grommet 12), is critical to successful and timely healing of the pierced opening. Said annular collar or flange 22 may be formed with ample rigidity and thickness for easy grasping for the above rotation purpose, while still maintaining a relatively thin cross section. Further, the shaft portion 24 of grommet 12 may be of ample length to allow the space needed for easy grasping of said flange 22. As mentioned above, when ornamental earrings such as depicted in FIG. 14 or hoops are inserted into the shaft 24 of grommet 12 said flange 22 will not be noticeable to casual observance as with prior art. Furthermore, said flange 22 may be further camouflaged by forming the flange 22 as clear or translucent or in any number of skin toned colors.

According to the preferred embodiment, the relatively soft material of said grommet shaft 24 may preferably provide the frictional means to retain the ornamental earring post 18 of earring 14 FIG. 1 with a press fit.

Optional means of facilitating a press fit of earring 14 into grommet 12e is described in FIG. 7 wherein the interior walls 38 of the grommet 12e are shown to be tapered to form a wedge effect 40. The normal compressive force of the surrounding earlobe or cartilage flesh about the grommet 12 will further indirectly aid in the desired press fit to retain the shaft 18 of ornamental jewelry 14, and thereby potentially eliminate the need for a standard jewelry “butterfly” clasp. As mentioned, the flexible walls of the healing grommet 12 will also allow for the insertion of curved jewelry such as hoops.

Alternatively, an ornamental earring may be retained within the healing grommet 12 by means of a standard clasp or clutch which may be snapped onto the distal end 20 of earring 14 after said earring 14 is inserted through the imbedded grommet 12. Said use of a standard clasp may serve the dual purpose of simultaneously retaining the grommet 12 in the ear opening.

To facilitate delivery of the novel healing grommet 12 of the present invention there is to be detachably mounted axially about the shaft portion 24 of the grommet 12 a beveled cannula member 10 having a hollow lumen extending there through. When assembled and then deployed the grommet 12 is to be cylindrically encased within the cannula 10. In this regard it is contemplated that when deployed, the cannula 10 will be advanced completely through the earlobe or ear cartilage tissue with the grommet 12 remaining embedded there within by virtue of the anchoring effect provided by the annular collar or flange 22 formed upon the proximal end of such grommet 12. Said pierced opening or pathway is produced by the beveled cutting edge 32 of said cannula 10. Although depicted as a beveled cannula, it should be recognized that cannula member 10 may take any of a variety of conventional cutting needle designs known in the medical arts to produce a clean cutting-edge incision through the ear tissue optimal to rapid healing, such as those produced by Hart Enterprises, Inc. of Wyoming, Mich. or later developed in the art. Advantageously, unlike prior art systems the cannula is a one-piece seamless cutting device. In contrast to prior art the cannula tip 32 may be ground or formed into any number of angles and shapes optimally customized for cutting pathways through different areas of the ear such as the earlobe or cartilage while avoiding the tearing effect of prior art tips. 35 Advantageously, unlike prior art systems, by using a razor cutting edge the tissue undergoes minimal tearing which is known in the art to occur by blunt or pointed piercing earrings or posts. Such prior art practices are known to produce significant trauma, swelling and recurring inflammation, as well as increase the chance of infection. Additionally, healing times are known to be substantially prolonged due to such prior art techniques.

Trauma is further minimized by virtue of the design of the present invention insofar as the detachable cannula portion 10 is preferably mounted completely about shaft portion 24 of the healing grommet 12 such that the same forms a continuous cylindrical body as the same is advanced through tissue (although it is likewise contemplated that cannula 10 may only cover a portion of shaft 24). Because the cannula member 10 may be hollow throughout there may be disposed within the cannula 10 gauze or the like to insulate and absorb such tissue, blood, etc. The cannula 10 and elongate portion 24 of the healing grommet 12 may additionally be coated or saturated with an anesthetic or anti-microbial agent, or both, or other healing agents as may be necessary for a given application. Although not shown, it is further contemplated that the healing grommet 12 and more particularly the cannula 10 mounted thereon, can minimize trauma even further to the extent the same is deployed such that the cannula 10 is simultaneously rotated while being axially advanced through the target site of tissue. In this regard, it is well known in the art that trauma is minimized to the extent a rotational shearing force is applied to increase a coring effect, if desirable, as has been established in medical biopsy procedures.

In another embodiment of the present invention the cannula 10a depicted in FIG. 2 may be formed similarly to the cannula 10 referenced above, except for having a closed distal end 32a. Advantageously, as with the above cannula member 10, the closed tip version 32 may accommodate any number of designs of sharpened tips customized to optimally cut a pathway through the ear for the same advantages cited above. With the closed tip design 32 the above mentioned tissue and blood cannot enter the cannula 10a from the distal end 32, and the above mentioned gauze or insulating material would not be needed.

Although a delivery device for implanting the cannula/healing grommet piercing assembly 10,12 is not shown it is contemplated that, as with conventional devices, the cannula/healing grommet assembly 10,12 would be forced or shot through the “gun barrel” of a disposable delivery device. Said barrel would be aligned with a cannula “catching” and receiving cartridge integrated into the device and situated on the opposite side of the ear tissue being pierced. Maintaining alignment of the cannula/healing grommet assembly 10,12 within said barrel and also through the targeted ear tissue, may be achieved by modifying the flange 22 of grommet 12 when piercing to take a cylindrical shape to conform to the inside of said delivery barrel. Additionally, the grommet assembly delivery device may provide for any of several means of alignment and guidance of the distal end 32 of the cannula 10. When the delivery barrel of the above device and the receiving cartridge are positively converged thus clamping against the opposite sides of the targeted ear, then alignment may be relatively easily assured. As soon as the cannula tip 32 pierces the ear tissue the cannula tip 32 becomes anchored into an additional point of guidance and alignment.

As will be appreciated, to enhance the safety during utilization of such novel piercing/healing earring system in FIG. 1 above, in particular to isolate the cannula member 10 intended to be mounted thereonto said healing grommet 12, it will be recognized that following the insertion of the healing grommet 12 through a given cross-section 24 of tissue it will be necessary to capture and isolate the cannula member 10 utilized to form the passageway there through. To that end, it is contemplated that a specialized cartridge, such as those disclosed in co-pending patent application Ser. No 09/264,498, (the teachings of which are expressly incorporated by reference), or other cartridge designed to retain and isolate a spent cannula, facilitate the isolation of the cannula member 10 utilized as part of the piercing process. In this respect, it should be recognized that such cartridge may function merely to isolate the cannula member 10. Said isolation is well known as a requirement under government regulations for handling medical waste commonly dispensed into well known SHARPS containers.

It has been mentioned that during the extended healing phase it is necessary to wear the piercing earring night and day. Said earrings are commonly retained with metal clasps which tend to scratch and irritate end users especially when said metal clasps are pressed against the side of the neck when reclining. Such irritation may be substantially or completely eliminated by the use of the novel healing grommet 12. It is first possible with the present invention to avoid the problem by simply removing the ornamental earring 14 and its accompanying clasp before sleeping. However, when end users prefer to leave ornamental earrings in place when reclining it is important to provide means for avoiding the use of hard-edged rear clasps, traditionally provided by current vendors, to retain the ornamental earring 14 and I or the grommet 12. If the end user consumer desires to leave the earring 14 in place while sleeping, then a soft rear clutch may be employed and attached to the distal end 20 of the ornamental earring 14. Alternatively, to protect the end user during sleep from the pointed end 20 of the earring 14 the retaining washer 34 depicted in FIG. 5 may be formed in a conical shape to insulate the sharp end 20 of the earring 14 while also retaining the grommet 12a at both ends of the pierced channel. Though not shown, another means for retaining the grommet 12 at both ends of the pierced channel, while providing a relatively comfortable backing when the end user is reclining, may be achieved by providing a shaft 24 that is sufficiently long enough to bend or fold after exiting the pierced channel.

In use it will be desirable to prevent the grommet 12 from being inadvertently pulled out of its implant position in the pierced channel when the end user removes the ornamental earring 14. Optional designs will continue to be discussed for positively retaining the grommet 12 at both ends of the pierced channel, and for adding frictional resistance to said removal of shaft 24 to overcome the degree of press fit used to hold the shaft 18 of earring 14 inside the shaft 24 of grommet 12.

In use, an easily learned practical means of preventing the grommet 12 from being inadvertently removed when removing ornamental jewelry such as 14, will be to simply apply moderate opposing pressure against the flange 22 of grommet 12 when removing said earring 14.

In another grommet embodiment an alternative means of retaining the shaft 18 of ornamental jewelry 14 is depicted in FIG. 1. By designing interior walls 38, FIG. 7 which taper and narrow such as depicted in the cross section of grommet 12e the shaft 18 of earring 14 may be impinged or wedged as it is advanced into the narrowing channel 40 of grommet 12e. Such impingement may provide the further friction press fit needed to retain the earring 14 and to do so advantageously without the need for a standard clasp.

Alternative embodiments of the preferred healing grommet 12 are depicted in FIGS. 3 through 6 depicting novel ways to prevent the grommet 12 from inadvertently slipping out of position once implanted and resting within a given pierced channel, and to also retain said grommet 12 without the need for conventional clasps.

In a preferred alternative grommet embodiment depicted in FIG. 3, a healing grommet is shown to be positively retained with a washer 34. In use, after the shaft of grommet 12a is implanted into the pierced opening washer 34 is to be slid onto and over the barbed distal end 26a of grommet 12a and onto the shaft 24a as depicted in FIG. 3, and said washer 34 will be retained by said barbs 29, and thereby preventing the healing grommet 12a from inadvertent removal. To facilitate sliding the washer 34 over the barbed end 26a and onto the shaft 24a of grommet 12a the inside ring 35 of the washer 34 may be formed of a thinner membrane than the outer section of the washer 34 50 the inner ring 35 may flex and yield as it is pushed over the tapered and barbed end 26a of the grommet shaft 24a. Alternatively, the inner ring 35 may be formed with slots or be otherwise keyed to the distal end 26a so as to freely slide past the end 26a and onto the shaft 24a. In the latter case in use after the washer 34 passed the barbed end the washer 34 would then immediately be rotated a very short distance to pass the keyed “doors” and thereby achieve the reasonably high probability of remaining locked in place on shaft 24a of grommet 12a. In a further alternative version not shown, washer 34 may be formed as a “C” clip designed to potentially automatically slide into a channel slot circumscribing the distal end 26a of the grommet post 24a.

Healing grommets may advantageously be formed to be self anchoring at both ends of the pierced channel. With a one-piece self anchoring version it is contemplated that the distal end of said grommet be integrally formed to have distal ends 26b, 26c of FIGS. 4 and 5 which are of larger diameters in their memory states than their posts 24b, 24c. To provide for easy insertion into the pierced opening the enlarged distal ends 26b, 26c will need to be reduced to a smaller diameter. FIG. 8 depicts one such reducing device which is described below. As described below there is anticipated a need for the end user consumer to replace healing grommets intermittently during the extended healing process, one reason being to introduce fresh anti-microbial agents to the pierced opening. The self anchoring grommet versions depicted in FIGS. 4 and 5 are well suited to insertion by the end user by employing the “jaw” or “V” clamp 16 depicted in FIG. 8, and same are referred to here as replacement grommets. Though not depicted in use replacement grommet/“jaw” clamp assemblies would be provided to the end user.

In said the alternative embodiment replacement grommet as depicted in FIG. 4, the bulbous memory state of the distal end 26b is depicted as closed. One potential novel advantage of a closed end 26b design is that said closed end 26b may potentially serve to block or otherwise prevent the hard and pointed distal end 20 of an ornamental earring 14, FIG. 1 from coming into direct contact with the side of the neck and cause the above noted irritation. In a further preferred alternative replacement grommet embodiment depicted in FIG. 5, there is provided a grommet 12c having a memory state taking the shape flared or trumpet-shaped distal end 26c which would impinge against the back side of the ear for the above self-anchoring purposes. The trumpet-shaped distal end 26c of grommet 12c is depicted as hollow throughout both ends of the grommet.

As both alternative replacement grommet embodiments of FIGS. 4 and 5 contain distal ends 26b, 26c which are larger than their respective shafts 24b, 24c, to facilitate insertion of said shafts 24b, 24c, it will be necessary to compress said distal ends 26b, 26c during insertion. In assembly, special fixtures may be readily made to compress the trumpet end 26c of grommet 12c, and the bulbous end 26b of grommet 12b to thus insert same into a “jaw” clamp 16 as depicted in FIG. 8 for storage and later deployment. As mentioned above it may become desirable to replace grommets intermittently during healing for the purpose of introducing fresh antimicrobial agents or for other purposes to enhance healing.

In use the trumpet grommets 12c and the bulbous grommets 12b, may be prefitted with an elongated “alligator” jaw or “V” type clamp 16 depicted in FIG. 8. As illustrated, such jaw clamp 16 has first and second arm members 42, 43, that impart an inwardly compressive force. In use, the arms 42, 43 compress about the flared trumpeted portion 26c, of grommet 12c, and compress about the bulbous portion 26b of grommet 12b to thus enable the same to assume a flattened, more radially compressed configuration to thus enable such portions 26c, 26b to be inserted directly through a pre-formed pierced passageway. The trumpet end 26c also depicts relief gaps or slits 27 to serve as gussets and facilitate compressing by the clamp 16 of FIG. 8. In the above scenario, end user customers would simply insert the jaw clamp 16 with the healing grommet engaged therein and pull the clamp 16 off from its exit site, as would occur when the clamp 16 is advanced through a piercing and then when the clamp 16 is removed leaving the grommet 12c in position. Although depicted as a jaw type clamp, it will be readily recognized by those skilled in the art that such clamp 16 may take the form of a conical sleeve or jacket which radially encases the distal end 26b, 26c of the grommets 12b, 12c, such that the same trumpet and bulbous distal ends 26b, 26c are caused to assume such compressed configuration unless otherwise unconstrained.

Grommet 12d of FIG. 6 depicts an alternative embodiment of a novel grommet retaining means for the purpose of further resisting inadvertent removal of the grommet 12 when ornamental earrings such as 14 are removed. The shaft 24d is depicted to be textured with sawtooth barbed ridges 36 oriented to facilitate entry into the pierced opening yet to resist inadvertent exiting from the pierced opening. Such barbs 36 could be alternatively formed as bumps or ridges or a combination of same. It is well known in the industry that the pierced opening conforms to the shape and contour of an implant. For example it is well known that the tissue surrounding pierced openings will close down to the smaller diameter of ornamental earring shafts after having been previously formed with a larger diameter piercing earring shaft. Therefore, the above described textures or barbs 36 will be predictably safe to use as well as effective.

Advantageously, by introducing the grommets of the present invention, there is thus substantially reduced, if not eliminated, the possibility of infection that can occur in those innumerable individuals impatient with the healing process and who want to rapidly change jewelry following the formation of a piercing, and prior to completion of initial healing. As is widely known prematurely changing jewelry following the formation of a piercing greatly increases the chances that an infection can develop at the piercing site, thus significantly prolonging healing time, and increasing customer frustration.

Although the invention has been described herein with specific reference to a presently preferred embodiment thereof, it will be appreciated by those skilled in the art that various modifications, deletions, and alterations may be made to such preferred embodiment without departing from the spirit and scope of the invention.

1. A system for forming a piercing and introducing a grommet through tissue to define an axial passageway therethrough comprising:
(a) a grommet member having proximal and distal ends, said grommet having an annular collar formed about said proximal end, said grommet having a lumen extending therethrough defining an axial passageway;
(b) a piercing mechanism detachably mounted axially about said grommet, said piercing mechanism being operative to form said piercing through said tissue such that said piercing mechanism becomes detaches from said grommet when said piercing mechanism extends through said tissue.

2. The system of claim 1 wherein said piercing mechanism comprises a cannula member having a sharpened bevel tip and a hollow lumen extending therethrough for housing said grommet member as said cannula is advanced through said tissue.

3. The system of claim 1 wherein said piercing mechanism comprises a closed-in stylet having a hollow lumen extending therethrough for housing said grommet as said piercing mechanism is advanced through said tissue.

4. The system of claim 1 wherein said grommet further includes an anchor mechanism formed on the distal end thereof to facilitate the ability of the grommet to remain resident within said piercing formed by said piercing mechanism.

5. The system of claim 4 wherein said anchor mechanism comprises a bulbous head operatively transitional between a first reduced configuration such that the anchor mechanism may be received within said piercing mechanism, and a second expanded configuration such that once said grommet is disposed within said piercing, said bulbous head assumes an expanded configuration operative to force said grommet to nest within said piercing.

6. The system of claim 4 wherein said anchor mechanism of said grommet comprises the combination of a barbed member formed upon said distal end of said grommet and a washer member positionable thereabout, said washer and barbed end cooperatively defining an anchor for said grommet when operatively coupled to one another.

7. The system of claim 4 wherein said distal end of said grommet comprises a flared, trumpet-shaped distal end operatively transitional between a first compressed configuration such that said distal end may be nested within said piercing mechanism and a second operative configuration whereby said distal end assumes said flared, trumpet shape, such flared, trumpet shape being operative to cause such grommet to remain nested within said piercing formed by said piercing member.

8. The system of claim 4 wherein said grommet defines a lumen extending therethrough, said lumen being operative to receive an earring post.

9. The system of claim 7 wherein said lumen comprises a generally conical or frustro-conical shape operative to impart a compressive force to said post to enable said post to remain firmly retained therein.

10. The system of claim 4 wherein said anchor mechanism comprises a multiplicity of barbs formed upon the length of said grommet, said barbs being operatively arranged such that the grommet is freely extensible through said piercing but resistant to movement in an opposed direction.
